(19) United States
(12) Patent Application Publication
(10) Pub. No.: US 2023/0045205 A1
(43) Pub. Date: Feb. 9, 2023

(54) HIGH-THROUGHPUT AUTOMATED STRAIN LIBRARY GENERATOR

(71) Applicant: Zymergen Inc., Emeryville, CA (US)

(72) Inventors: Sara da Luz Areosa CLETO, Emeryville, CA (US); Philip D. WEYMAN, Alameda, CA (US)

(21) Appl. No.: 17/959,679

(22) Filed: Oct. 4, 2022

Related U.S. Application Data
(63) Continuation of application No. PCT/US2021/026041, filed on Apr. 6, 2021.
(60) Provisional application No. 63/006,999, filed on Apr. 8, 2020.

(51) Int. Cl. C12N 15/10

(52) U.S. Cl. CPC C12N 15/1093; C12N 15/1089

(57) ABSTRACT

A method for automated, high throughput cellular library generation is disclosed. The method includes providing a suspension including transformed cells and plating the transformed cells onto solid surfaces of each of at least one reservoir of a reservoir plate. The solid surfaces can include a liquid growth medium. The reservoir plate is incubated, and after cellular growth has occurred on at least one plated surface of the reservoir plate, a series of automatic steps are performed. The automatically-performed steps include adding disaggregation solution to the reservoir plate, applying a mechanical force, such as a rotational force, to the reservoir plate to produce resuspended cells, and/or collecting the resuspended cells.

### CROSS-REFERENCE TO RELATED APPLICATIONS

This application is a continuation of International Patent Application No. PCT/US2021/026041, filed Apr. 6, 2021 and titled “High-Throughput Automated Strain Library Generator,” which claims priority to and the benefit of U.S. Provisional Patent Application No. 63/006,999, filed Apr. 8, 2020 and titled “High-Throughput Automated Strain Library Generator” the contents of each of the aforementioned applications are incorporated by, reference herein in their entireties for all purposes.

### FIELD OF THE DISCLOSURE

The present disclosure generally relates to systems and methods for the improvement of natural product discovery. The disclosed systems and methods facilitate high-throughput generation of cellular libraries that preserve library diversity, and downstream processing.

### BACKGROUND

Known methods of generating and collecting pools of cells containing different molecules of DNA in their chromosome or episome include plating of the cells to a solid medium, and subsequent resuspension of the cells via either a human-mediated step to scrape the resulting colonies of cells, or use of an automated colony picking device. These resuspension methods are time-consuming and prone to cross-contamination and sample loss, and colony picking requires specialized and expensive laboratory equipment and can introduce size bias (e.g., such that larger colonies are favored).

There is an ongoing and unmet need for methods, systems, and tools for preserving library diversity while still allowing automated and HTP capabilities.

### SUMMARY OF THE DISCLOSURE

In some embodiments, a method for automated, high throughput cellular library generation includes providing a suspension including transformed cells and plating the transformed cells onto solid surfaces of each of at least one reservoir of a reservoir plate. The solid surfaces of each of at least one reservoir of a reservoir plate can include a liquid growth medium. The reservoir plate is incubated, and after cellular growth has occurred on at least one plated surface of the reservoir plate, a series of steps are performed automatically. The automatically-performed steps include one or more of: adding disaggregation solution to the reservoir plate, applying a mechanical force to the reservoir plate to produce resuspended cells, and collecting the resuspended cells. The mechanical force can be, for example, a rotational force between 200 revolutions per minute (RPM) and 3,000 RPM. The mechanical force can be applied for a duration, for example, of between 10 seconds and 10 minutes, or between 30 seconds and 60 seconds. The automatically-performed steps can also include transferring the collected resuspended cells to a multi-well plate after the cellular growth has occurred on at least one plated surface of the reservoir plate. The method can also include introducing DNA to cells to produce the transformed cells, prior to the plating. Alternatively or in addition, the method can also include waiting a predetermined recovery duration after producing the transformed cells and prior to the plating. Alternatively or in addition, the method can also include processing the collected resuspended cells. The at least one reservoir of the reservoir plate can include, for example, a relatively small number of reservoirs (also referred to herein as “wells”), for example 1 reservoir or 2 reservoirs (e.g., if a larger surface is needed, such as may be the case for mammalian cells), or a larger number of reservoirs, for example 6 reservoirs, 12 reservoirs, 24 reservoirs, 96 reservoirs, or 384 reservoirs.

In some embodiments, for example when a liquid growth medium is used, the reservoir plate is not prepared in advance of the plating. In other embodiments, the reservoir plate is prepared prior to the plating, for example by applying a predetermined volume of a. growth medium into each well of the reservoir plate.

In some embodiments, the method for automated, high throughput cellular library generation also includes preserving the collected resuspended cells at a predetermined temperature, for example at −80° C., or at −130° C. (e.g., mammalian cells), or at −20° C. (e.g., for applications in which cell viability is not a priority), or between −130° C. and −20° C., or between −80° C., and −20° C., or between −130° C. and −80° C.

In some embodiments, after the plating of the transformed cells, each reservoir from the reservoir plate includes a different associated plasmid.

The disaggregation solution can include at least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include a viscous liquid. Alternatively or in addition, the disaggregation solution includes at least one of a positive selection agent or a negative selection agent. Alternatively or in addition, the disaggregation solution includes a microbial growth medium, glycerol, and an antibiotic.

In some embodiments, after the cellular growth has occurred on the at least one plated surface of the reservoir plate, and prior to applying the mechanical force to the reservoir plate, beads are added to the reservoir plate.

In some embodiments, the method also includes controlling a temperature of the reservoir plate during the application of the mechanical force, such that the temperature of the reservoir plate is between 4° C. and 40° C.

In some embodiments, a system for performing automated, high throughput cellular library generation includes a processor, a workstation, and at least one reservoir plate (e.g., including a plurality of wells). Optionally, the system also includes a plurality of liquid sensing tips.

The workstation includes a robotic arm (e.g., including a multiple-channel pipetting head) and a high-speed shaker. The reservoir plate(s) includes a growth-enabling matrix. The processor is configured, during operation, to send a signal to control the robotic arm to cause transfer of a suspension including transformed cells to each of at least one reservoir of the reservoir plate. The processor is also configured, during operation, to detect that cellular growth has occurred on at least one plated surface of the reservoir plate, and in response to detecting the cellular growth, send a signal to cause introduction of a disaggregation solution to the reservoir plate, and apply a mechanical force (e.g., orbital shaking forces), using the high-speed shaker, to the reservoir plate to produce resuspended cells. The disaggregation solution can include a viscous liquid. Alternatively or in addition, the disaggregation solution can include at least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include at least one of a positive selection agent or a negative selection agent. Alternatively or in addition, the disaggregation solution can include a microbial growth medium, glycerol, and an antibiotic. The mechanical force can include a rotational force between 200 RPM and 3,000 RPM, and/or can be applied for a predetermined duration e.12., between 10 seconds and 10 minutes, or between 30 seconds and 60 seconds). The processor can also be configured, during operation, to send a signal to cause collection of the resuspended cells from the reservoir plate. Optionally, the processor can be further configured to wait a predetermined incubation period prior to detecting the predetermined amount of cellular growth.

In some embodiments, the processor is further configured to automatically transfer, after the cellular growth has occurred on at least one plated surface of the reservoir plate, the collected resuspended cells to a multi-well plate.

In some embodiments, the processor is further configured to control a temperature of the reservoir plate during the application of the mechanical force, such that the temperature of the reservoir plate is between 4° C. and 40° C.

In some embodiments, a method of removing a plurality of discrete colonies from solid supports of a reservoir plate includes controlling a robotic arm to cause transfer of a suspension including a population of cells to each of a plurality of reservoirs of the reservoir plate. The method also includes determining, after transferring the suspension to the reservoir plate, that each discrete colony from the plurality of discrete colonies has grown on an associated solid support of an associated reservoir from the plurality of reservoirs of the reservoir plate. In response to detecting the cellular growth, a disaggregation solution is introduced to the reservoir plate, and a mechanical force is applied to the reservoir plate to produce resuspended cells within each reservoir from the plurality of reservoirs of the reservoir plate. The disaggregation solution can include a viscous liquid. Alternatively or in addition, the disaggregation solution can include at least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include at least one of a positive selection agent or a negative selection agent. Alternatively or in addition, the disaggregation solution can include a microbial growth medium, glycerol, and an antibiotic. The mechanical force can include a rotational force between 200 RPM and 3,000 RPM, and/or can be applied for a predetermined duration (e.g., between 10 seconds and 10 minutes, or between 30 seconds and 60 seconds).

The method optionally also includes performing an automated library preparation using the resuspended cells. The method optionally also includes adding beads to the reservoir plate prior to applying the mechanical force to the reservoir plate.

In some embodiments, the method also includes controlling a temperature of the reservoir plate during the application of the mechanical force, such that the temperature of the reservoir plate is between 4° C. and 40° C.

In some embodiments, a method includes providing a suspension including a population of cells (e.g, transformed cells), plating cells from the population of cells onto solid surfaces of each of at least one reservoir of a reservoir plate, and incubating the reservoir plate. After cellular growth has occurred on at least one plated surface of the reservoir plate, the method also includes automatically adding disaggregation solution to the reservoir plate, automatically applying a mechanical force to the reservoir plate to produce resuspended cells, and automatically collecting the resuspended cells. The disaggregation solution can include a. viscous liquid. Alternatively or in addition, the disaggregation solution can include at least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include at least one of a positive selection agent or a negative selection agent. Alternatively or in addition, the disaggregation solution can include a microbial growth medium, glycerol, and an antibiotic. The mechanical force can include a rotational force between 200 RPM and 3,000 RPM, and/or can be applied for a predetermined duration (e.g., between 10 seconds and 10 minutes, or between 30 seconds and 60 seconds).

In some embodiments, the population of cells includes culturable bacteria. In other embodiments, the population of cells includes at least one of: edited microbes, or natural microbes.

In some embodiments, the method also includes automatically transferring the collected resuspended cells to a multi-well plate after the cellular growth has occurred on at least one plated surface of the reservoir plate.

In some embodiments, the method also includes performing an automated library preparation using the collected resuspended cells.

In some embodiments, the method also includes adding beads to the reservoir plate prior to applying the mechanical force to the reservoir plate.

In some embodiments, the method also includes controlling a temperature of the reservoir plate during the application of the mechanical force, such that the temperature of the reservoir plate is between 4° C. and 40° C.

In some embodiments, a method includes providing a suspension including a population of transfected cells (e.g., mammalian cells), transferring the population of transfected cells to at least one reservoir of a reservoir plate, and incubating the reservoir plate. After cellular growth has occurred within the reservoir plate, the method further includes automatically adding disaggregation solution to the reservoir plate, automatically applying a. mechanical force to the reservoir plate to produce resuspended cells, and automatically collecting the resuspended cells. The disaggregation solution can include a viscous liquid. Alternatively or in addition, the disaggregation solution can include at least one of: Triton 2100, Tween-80, Urea, Pluronic™ F-68Accumax, Accutase, Lipase, a detergent, a protease, an amilase, a cellulose, or a glycerol. Alternatively or in addition, the disaggregation solution can include at least one of a positive selection agent or a negative selection agent. Alternatively or in addition, the disaggregation solution can include a microbial growth medium, glycerol, and an antibiotic. The mechanical force can include a rotational force between 200 RPM and 3,000 RPM, and/or can be applied for a predetermined duration (e.g., between 10 seconds and 10 minutes, or between 30 seconds and 60 seconds).

In some embodiments, the method also includes performing an automated library preparation based on the collected resuspended cells.

In some embodiments, the method also includes adding beads to the reservoir plate prior to applying the mechanical force to the reservoir plate.

In some embodiments, the method also includes controlling a temperature of the reservoir plate during the application of the mechanical force, such that the temperature of the reservoir plate is between 4° C. and 40° C.

In some embodiments, a spore production and harvesting method includes providing a suspension including a population of cells, plating cells from the population of cells onto solid surfaces of each of at least one reservoir of a reservoir plate, and incubating the reservoir plate. The solid surfaces of the at least one reservoir of the reservoir plate can include, for example, an antibiotic. After cultures have sporulated on at least one plated surface of the reservoir plate, the method also includes automatically adding disaggregation solution to the reservoir plate, automatically applying a mechanical force to the reservoir plate to produce a spore suspension including at least a portion of the spores, and automatically collecting the spore suspension. The disaggregation solution can include a viscous liquid. Alternatively or in addition, the disaggregation solution can include at least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include at least one of a positive selection agent or a negative selection agent. Alternatively or in addition, the disaggregation solution can include a microbial growth medium, glycerol, and an antibiotic. The mechanical force can include a rotational force between 200 RPM and 3,000 RPM, and/or can be applied for a predetermined duration (e.g., between 10 seconds and 10 minutes, or between 30 seconds and 60 seconds).

In some embodiments, the method also includes performing an automated library preparation based on the spore suspension.

In some embodiments, the method also includes adding beads to the reservoir plate prior to applying the mechanical force to the reservoir plate.

In some embodiments, the method also includes controlling a temperature of the reservoir plate during the application of the mechanical force, such that the temperature of the reservoir plate is between 4° C. and 40° C.

### BRIEF DESCRIPTION OF THE SEVERAL VIEWS OF THE DRAWINGS

FIG. 1 is a diagram showing a system for performing automated, high throughput cellular library generation, according to some embodiments.

FIG. 2 is a flow diagram showing a method for automated, high throughput cellular library generation, according to some embodiments.

FIG. 3 is a flow diagram showing a method of removing a plurality of discrete colonies from solid supports of a reservoir plate, according to some embodiments.

FIG. 4 is an overview of a library generation process, according to some embodiments.

FIG. 5 is an outline of an example application of the process of FIG. 4.

FIG. 6 shows example outcomes of a colony resuspension process, according to some embodiments.

### DETAILED DESCRIPTION

### Definitions

While the following terms are believed to be well understood by one of ordinary skill in the art, the following definitions are set forth to facilitate explanation of the presently disclosed subject matter.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein the terms “cellular organism” “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “cellular organisms” or “microbes” of lists/tables and figures present in the disclosure. This characterization can refer to not only the identified taxonomic genera of the tables and figures, but also the identified taxonomic species, as well as the various novel and newly identified or designed strains of any organism in said tables or figures. The same characterization holds true for the recitation of these terms in other parts of the Specification, such as in the Examples.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane): extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., bacteria, yeast cell, fungal cell., CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived. It is understood that in some embodiments, the terms refer not only to the particular recombinant host cell in question, but also to the progeny or potential progeny of such a host cell.

The term “wild-type: microorganism” or “wild-type host cell” describes a cell that occurs in nature, i.e. a cell that has not been genetically modified.

The term “genetically engineered” may refer to any manipulation of a host cell's genome (e.g. by insertion, deletion, mutation, or replacement of nucleic acids).

The term “control” or “control host cell” refers to an appropriate comparator host cell for determining the effect of a genetic modification or experimental treatment. In some embodiments, the control host cell is a wild type cell. In other embodiments, a control host cell is genetically identical to the genetically modified host cell, save for the genetic modification(s) differentiating the treatment host cell. In some embodiments, the present disclosure teaches the use of parent strains as control host cells (e.g., the S₁ strain that was used as the basis for the strain improvement program). In other embodiments, a host cell may be a genetically identical cell that lacks a specific promoter or SNP being tested in the treatment host cell.

As used herein, the term “allele(s)” means any of one or more alternative forms of a gene, all of which alleles relate to at least one trait or characteristic. In a diploid cell, the two alleles of a given gene occupy corresponding loci on a pair of homologous chromosomes.

As used herein, the term “locus” (loci plural) means a specific place or places or a site on a chromosome where for example a gene or genetic marker is found.

As used herein, the term “genetically linked” refers to two or more traits that are co-inherited at a high rate during breeding such that they are difficult to separate through crossing,

A “recombination” or “recombination event” as used herein refers to a chromosomal crossing over or independent assortment.

As used herein, the term “phenotype” refers to the observable characteristics of an individual cell, cell culture, organism, or group of organisms Which results from the interaction between that individual's genetic makeup (i.e., genotype) and the environment.

As used herein, the term “chimeric” or “recombinant” when describing a nucleic acid sequence or a protein sequence refers to a nucleic acid, or a protein sequence, that links at least two heterologous polynucleotides, or two heterologous polypeptides, into a single macromolecule, or that re-arranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (h) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “endogenous” or “endogenous gene,” refers to the naturally occurring gene, in the location in which it is naturally found within the host cell genome. In the context of the present disclosure, operably linking a heterologous promoter to an endogenous gene means genetically inserting a heterologous promoter sequence in front of an existing gene, in the location where that gene is naturally present. An endogenous gene as described herein can include alleles of naturally occurring genes that have been mutated according to any of the methods of the present disclosure.

As used herein, the term “exogenous” is used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulator element can be prepared by isolating a portion of one of the polynucleotides of the disclosure that comprises the genetic regulatory element and assessing activity as described herein. Similarly, a portion of a polypeptide may be 4 amino acids, 5 amino acids. 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zang et al. (1997) PNAS 94:4504-4509; Crameri et al. (1998) Nature 391:288-291; and U.S. Pat. Nos. 5,605,793 and 5,837,458.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3^{rd} ed., Cold Spring Harbor Laboratory Press, Plainview, N.Y.). See also Innis et al., eds. (1990) PCR Protocols: A Guide to Methods and Applications (Academic Press, New York); Innis and Gelfand, eds, (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

As used herein, “promoter” refers to a DNA sequence capable of controlling the expression of a coding sequence or functional RNA. In some embodiments, the promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

“Operably linked” means in this context the sequential arrangement of the promoter polynucleotide according to the disclosure with a further oligo- or polynucleotide, resulting in transcription of said further polynucleotide.

The term “product of interest” or “biomolecule” as used herein refers to any product produced by microbes from feedstock. In some cases, the product of interest may be a small molecule, enzyme, peptide, amino acid, organic acid, synthetic compound, fuel, alcohol, etc. For example, the product of interest or biomolecule may be any primary or secondary extracellular metabolite. The primary metabolite may be, inter alia, ethanol, citric acid, lactic acid, glutamic acid, glutamate, lysine, threonine, tryptophan and other amino acids, vitamins, polysaccharides, etc. The secondary metabolite may be, inter alia, an antibiotic compound like penicillin, or an immunosuppressant like cyclosporin A, a plant hormone like gibberellin, a statin drug like lovastatin, a fungicide like griseofulvin, etc. The product of interest or biomolecule may also be any intracellular component produced by a microbe, such as: a microbial enzyme, including: catalase, amylase, protease, pectinase, glucose isomerase, cellulase, hemicellulase, lipase, lactase, streptokinase, and many others. The intracellular component may also include recombinant proteins, such as: insulin, hepatitis B vaccine, interferon, granulocyte colony-stimulating factor, streptokinase and others.

The term “carbon source” generally refers to a substance suitable to be used as a source of carbon for cell growth. Carbon sources include, but are not limited to, biomass hydrolysates, starch, sucrose, cellulose, hemicellulose, xylose, and lignin, as well as monomeric components of these substrates. Carbon sources can comprise various organic compounds in various forms, including, but not limited to polymers, carbohydrates, acids, alcohols, aldehydes, ketones, amino acids, peptides, etc. These include, for example, various monosaccharides such as glucose, dextrose (D-glucose), maltose, oligosaccharides, polysaccharides, saturated or unsaturated fatty acids, succinate, lactate, acetate, ethanol, etc., or mixtures thereof. Photosynthetic organisms can additionally produce a carbon source as a product of photosynthesis. In some embodiments, carbon sources may be selected from biomass hydrolysates and glucose.

The term “feedstock” is defined as a raw material or mixture of raw materials supplied to a microorganism or fermentation process from which other products can be made. For example, a carbon source, such as biomass or the carbon compounds derived from biomass are a feedstock for a microorganism that produces a product of interest (e.g. small molecule, peptide, synthetic compound, fuel, alcohol, etc.) in a fermentation process. However, a feedstock may contain nutrients other than a carbon source.

The term “volumetric productivity” or “production rate” is defined as the amount of product formed per volume of medium per unit of time. Volumetric productivity can be reported in gram per liter per hour (a/L/h).

The term “specific productivity” is defined as the rate of formation of the product. Specific productivity is herein further defined as the specific productivity in gram product per gram of cell dry weight (CDW) per hour (g/g CDW/h). Using the relation of CDW to OD₆₀₀ for the given microorganism specific productivity can also be expressed as gram product per liter culture medium per optical density of the culture broth at 600 nm (OD) per hour (g/L/h/OD).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product.

The term “titre” or “titer” is defined as the strength of a solution or the concentration of a substance in solution. For example, the titre of a product of interest (e.g. small molecule, peptide, synthetic compound, fuel, alcohol, etc.) in a fermentation broth is described as g of product of interest in solution per liter of fermentation broth (g/L).

The term “total titer” is defined as the sum of all product of interest produced in a process, including but not limited to the product of interest in solution, the product of interest in gas phase if applicable, and any product of interest removed from the process and recovered relative to the initial volume in the process or the operating volume in the process

As used herein, the term “HTP genetic design library” or “library” refers to collections of genetic perturbations according to the present disclosure. In some embodiments, the libraries of the present invention may manifest as i) a collection of sequence information in a database or other computer file, ii) a collection of genetic constructs encoding for the aforementioned series of genetic elements, or iii) host cell strains comprising said genetic elements. In some embodiments, the libraries of the present disclosure may refer to collections of individual elements (e.g., collections of promoters for PRO swap libraries, or collections of terminators for STOP swap libraries). In other embodiments, the libraries of the present disclosure may also refer to combinations of genetic elements, such as combinations of promoter:genes, gene:terminator, or even promoter:gene:terminators. In some embodiments, the libraries of the present disclosure further comprise meta data associated with the effects of applying each member of the library in host organisms. For example, a library as used herein can include a collection of promoter:gene sequence combinations, together with the resulting effect of those combinations on one or more phenotypes in a particular species, thus improving the future predictive value of using said combination in future promoter swaps.

As used herein, the term “SNP” refers to Small Nuclear Polymorphism(s). In some embodiments, SNPs of the present disclosure should be construed broadly, and include single nucleotide polymorphisms, sequence insertions, deletions, inversions, and other sequence replacements. As used herein, the term “non-synonymous” or non-synonymous SNPs” refers to mutations that lead to coding changes in host cell proteins

A “high-throughput (HTP)” method of genomic engineering may involve the utilization of at least one piece of automated equipment (e.g. a liquid handler or plate handler machine) to carry out at least one step of said method.

### Overview

Current state of the art methods for generating and collecting pools of cells containing different molecules in their chromosome or episome (referred to as “library generation”) involve plating to a solid medium and subsequent resuspension of the cells, with either a human-mediated step to manually (i.e., by hand) scrape the resulting colonies of cells, or with the use of cell scrapers or an automated colony picking device. For example, automated colony picking robots can be used to pick a required number of colonies, grow them in multi-well liquid plates and then combine them to mix the library into a single well. Automated colony picking methods have several drawbacks, including time consuming picking protocols (especially for libraries where millions of colonies are required), wasted consumables, expensive automation robotics and a potential colony-size bias that could lead to the exclusion of smaller-sized colonies.

In some known library generation methods, after introducing a diversity of DNA into cells, selection for transformed cells is performed. If the selection is performed in a liquid median, members of the library having the fastest growth rate may rapidly overtake the culture and dominate the library (a phenomenon sometimes known as “jackpotting”). This can lead to a lack of diversity and poor quality libraries. As a result, known library generation techniques rely upon plating transformed cells on solid medium. The problem with such techniques is that they have traditionally been resistant to automation. Although plating can be effectively automated, the scraping or resuspension of cells has not been successfully automated. Such methods can therefore be time-consuming, labor-intensive, and prone to error, and can require specialized and expensive laboratory equipment. No current commercially-available laboratory robot has the ability to grasp a cell scraper or similar tool, or sense solid surfaces and apply the appropriate force for cell scraping. The inventors are unaware of any existing method that allows for the efficient automated removal of cells or colonies from solid surfaces for downstream processing.

The presently disclosed inventions overcome these drawbacks to existing technology, and provide methods, systems, and tools for fully automated, high-throughput (HTP) cellular library generation. Embodiments set forth herein do not require human intervention, are compatible with liquid handling robotic workstations, and more cost-effective and time-efficient than known library generation techniques, while preserving library diversity. Methods set forth herein, in some embodiments, involve the use of workstations that include a robotic arm, a high-speed plate shaker, and 6-well reservoir plates containing a growth-enabling matrix such as agar. Methods set forth herein also avoid the need for colony picking. Applications of the present disclosure can include, but are not limited to: phenotype screening, enzymatic activity screening, next-generation sequencing (NGS), and plasmid assembly.

In some embodiments, an automated, HTP method to create cellular libraries, while preserving library diversity, includes plating transformed cells containing the library on solid surfaces. After plating and growth, libraries are returned, in an automated manner, to liquid format in 12-well, 24-well, 96-well or 384-well plates, or equivalent, such that the downstream processing of the strain library occurs in a high throughput manner.

In some embodiments, to ensure the preservation of cellular library diversity, agar-containing surfaces are used for plating said libraries. Agar-containing surfaces can be prepared, for example, by pouring a given volume (e.g. 8 milliliters (mL)) of melted agar-containing growth medium with one or more growth stimulation and/or repression additives into each well of a reservoir plate (e.g., including 6 wells or any number of wells in a higher or lower density). Upon solidification (e.g., after the agar surfaces of the wells have dried), cells suspended in a liquid and containing a diversity of chromosomes or episomes are added to the wells of the reservoir plate and allowed to grow on the agar surfaces of each well. Examples of known plating techniques are provided in “Plating and Transferring Cosmid and Plasmid Libraries,” by John H. Weis, Current Protocols in Molecular Biologies, Vol. 68, Issue 1 (October 2004) and “Aseptic Laboratory Techniques: Plating Methods,” by E. R. Sanders, Microbiology, Immunology, and Molecular Genetics, Issue 63 (May 2012), the entire contents of each of which are incorporated herein by reference in their entireties. The reservoir plate is incubated at an appropriate temperature, and when growth is visible a disaggregation or resuspension solution is added to each well of the plate. The reservoir plate is then subjected to orbital shaking forces for removal of the colonies of cells growing on the agar surface. After the orbital shaking forces have been applied, the resuspended cells are collected and either further processed or directly preserved at −80 C.

In some embodiments, cell and/or colony scraping from solid surfaces is performed in an automated fashion, and the scraped cells, once in suspension, can be collected and returned to multi-well plates compatible with HTP liquid handling systems for downstream processing, thereby facilitating HTP generation of cellular libraries with high diversity. Embodiments of the present disclosure can provide time and cost benefits for a variety of applications. For example, microbial strains are often engineered to carry specific mutations in their chromosome or episome. In an industrial setting, these are typically created in an HTP manner, resulting in tens of thousands of strains that need to be genotyped at any given time. In the cases where the strains have been barcoded and must be grown prior to genotyping, this step can be performed according to embodiments set forth herein, in a way that minimizes bias in the library while also allowing HIP functionality. Following growth and biomass retrieval, the samples can be lysed and used as template for a multiplex, PCR-based enrichment step to be sequenced by Next Generation Sequencing.

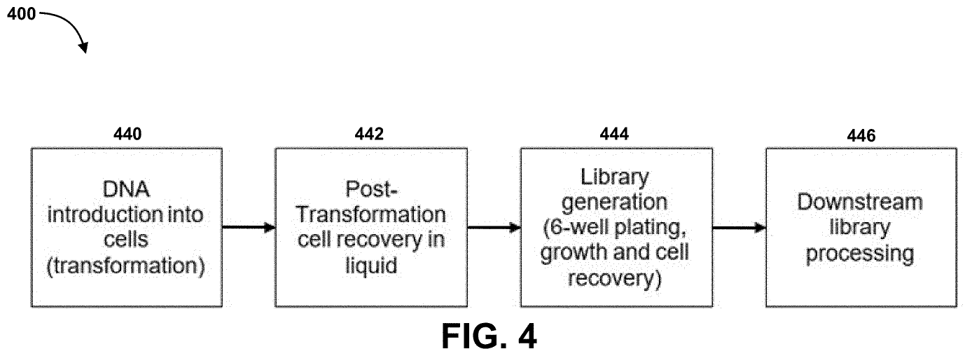

In some embodiments, libraries of strains are created by transformation, transfection, transduction or any other method that allows for the introduction of foreign DNA into cells. This can occur in a reservoir plate having a 96-well format, or any other format that allows for HTP processing of samples. The contents of the wells or aliquots thereof are then transferred via an automated protocol to wells of, for example, 6-well reservoir plates. Growth of the transformed cells on the solid matrix allows for the preservation of library diversity. After growth, cells grown on a solid surface (e.g., agar) are resuspended into liquid via an automated protocol, and transferred back to a 96-well plate for further processing of the library (e.g., sequencing, phenotypic screening, etc.), as shown and discussed with reference to FIG. 4, below.

As described herein, in accordance with some embodiments, a library of strains can refer to a library of strains where each strain in the library has a modification in its chromosome. Alternatively or in addition, a library of strains can refer to a library of strains where each strain in the library has a different plasmid/episome bearing a variant of an enzyme, gene, etc. Alternatively or in addition, a library of strains can refer to a library of strains where each strain in the library has a different plasmid/episome with a different gene/enzyme. The library of strains can be subsetted such that partial libraries are found in each of the wells of the original reservoir plate (e.g., a 96-well reservoir plate), for example to further reduce bias in the library. Strain libraries set forth herein can be made in microbial cells (bacterial, fungal, protist) or cultured higher eukaryotes (mammalian, insect, plant, etc.).

As described herein, in accordance with some embodiments, a reservoir plate can be a deep plate With a quantity of wells (e,g., between 1 and 12 wells, or 6 wells). The reservoir plate can include wells having high walls (or “deep wells”) and be divided into a certain number of parts. The bottom of each well (also referred to herein as a “division”) can contain a matrix, whether liquid or solid, that facilitates the growth of cells. The high walls allow for the HTP process to be executed without the risk of cross-contamination when, after a disaggregation solution is added to each well, the reservoir plate is shaken at high revolutions per minute (RPM) to cause release or liberation of the cells from the matrix and disaggregation of co-aggregated cells. The shape of the reservoir plate can be compatible with, and used with, a liquid handling robotic platform and robotic manipulator arms. The structure of the reservoir plate can allow for the reservoir plate to be shaken at high RPM without the loss of its physical integrity. The wells of the reservoir plate (e.g., the bottoms of the wells) can be of a plastic composition, and can be coated with agar or a similar nutritious matrix. The plastic bottoms of the well, in the presence of a nutritious liquid or solid, can support the growth of cells.

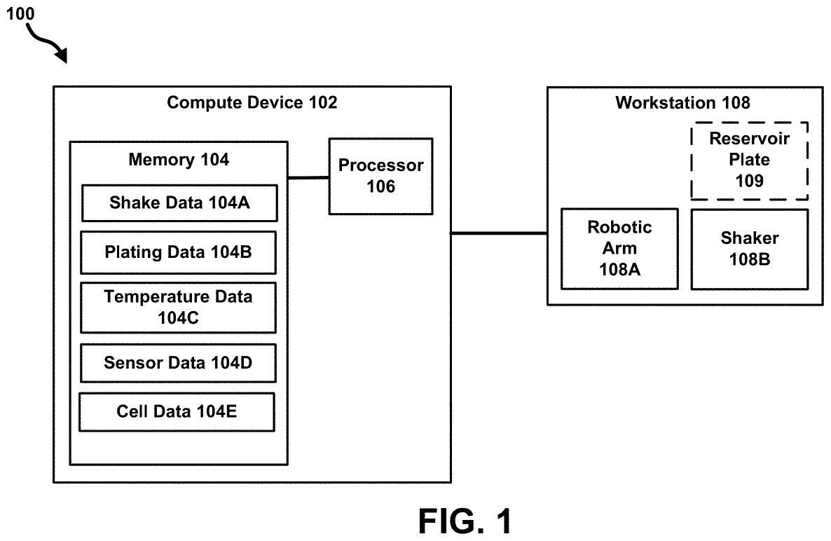

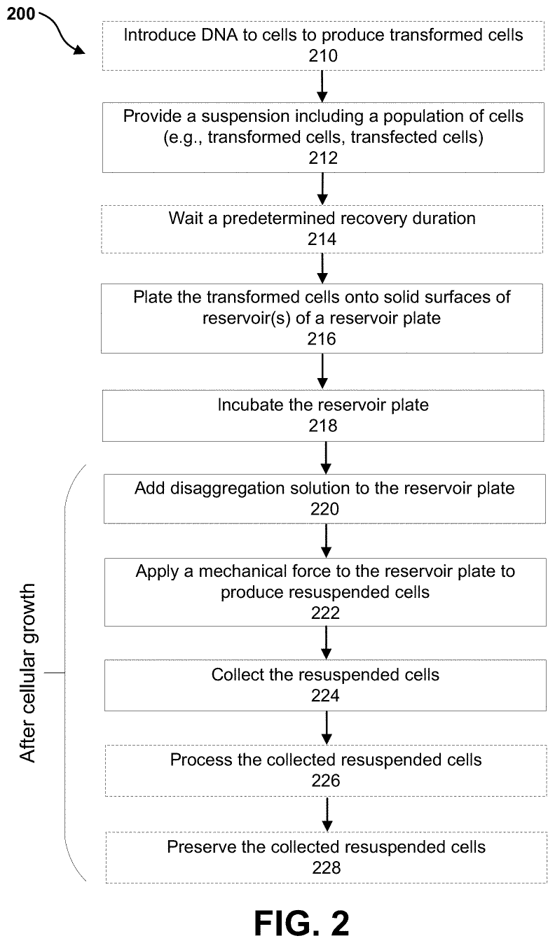

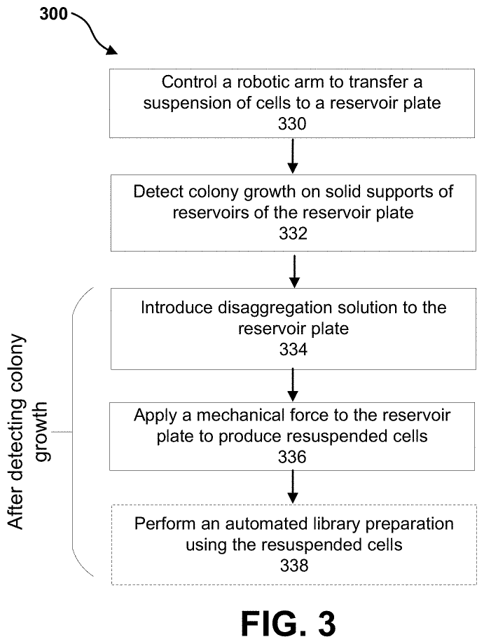

FIG. 1 is a diagram showing a system for performing automated, HTP cellular library generation, according to some embodiments. As shown in FIG. 1, the system 100 includes a compute device 102 and a workstation 108. The workstation 108 includes a robotic arm 108A (or one or more other liquid handling robotic platforms, e,g., with a flexible arm) and a high-speed shaker 108 (or “shaking platform”). The liquid handling robotic platform(s) can include a robotic pipetting head (e.g., a multiple-channel pipetting head, such as a 96-channel pipetting head) and/or liquid sensing tips. During operation of the system 100, one or more reservoir plates 109 (e.g., each including one well or a plurality of wells, the wells including a growth-enabling matrix) can be received by the workstation, for example such that the reservoir plate(s) 109 are disposed atop, or are otherwise in mechanical contact with, the shaker 108B. The compute device 102 includes a memory 104 in operable communication with a processor 106. The memory 104 can store data and processor-executable instructions (“code”) that can be executed by the processor 106, during operation of the system 100, for example to perform the methods shown and described with reference to FIGS. 2 and 3, discussed below. The data stored by the memory 104 can include one or more of: shake data 104A (e.g., parameter setpoints for the shaker 108B), plating data 104B temperature data 104C, sensor data 104D, cell data 104E, and/or any other data relevant to the execution of the processor-executable instructions.

The processor 106 is configured, during operation and by executing processor-executable instructions, to send a signal to control the robotic arm 108A to cause transfer of a suspension including transformed cells to each of at least one reservoir of the reservoir plate 109. The processor 106 is also configured, during operation, to detect that cellular growth has occurred on at least one plated surface of the reservoir plate 109, and in response to detecting the cellular growth, send a signal to cause introduction of a disaggregation solution to the reservoir plate 109, and apply a mechanical force (e.g., orbital shaking forces), using the high-speed shaker 108B, to the reservoir plate 109 to produce resuspended cells. The disaggregation solution can include a viscous liquid. Alternatively or in addition, the disaggregation solution can include at least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include at least one of a positive selection agent or a negative selection agent. Alternatively or in addition, the disaggregation solution can include a microbial growth medium, glycerol, and an antibiotic. The mechanical force applied by the high-speed shaker 108B can include a rotational force between 200 RPM and 3,000 RPM, and/or can be applied for a predetermined duration (e.g., between 10 seconds and 10 minutes, or between 30 seconds and 60 seconds). The processor 106 can also be configured, during operation, to send a signal to cause collection of the resuspended cells from the reservoir plate 109. Optionally, the processor 106 can be further configured to wait a predetermined incubation period prior to detecting the predetermined amount of cellular growth.

In some embodiments, the processor 106 is further configured to automatically transfer, after the cellular growth has occurred on at least one plated surface of the reservoir plate 109, the collected resuspended cells to a multi-well plate.

In some embodiments, the processor 106 is further configured to control a temperature of the reservoir plate 109 during the application of the mechanical force, such that the temperature of the reservoir plate 109 is between 4° C. and 40° C.

Although the system 100 of FIG. 1 is shown and described to include a robotic arm 108A and a shaker 108B, in other embodiments a system for performing automated, low throughput cellular library generation does not include a shaking platform and/or does not include a liquid handling robotic platform. For example, a system for performing automated, high throughput cellular library generation can include a processor, a workstation, and at least one reservoir plate (e.g., including a plurality of wells). Optionally, the system also includes a plurality of liquid sensing tips.

### Organisms Amenable to Genetic Design

The disclosed system for performing automated, HTP cellular library generation is applicable to any host cell organism where desired traits can be identified in a population of genetic mutants. Thus, as used herein, the term “microorganism” should be taken broadly. It includes, but is not limited to, the two prokaryotic domains, Bacteria and Archaea, as well as certain eukaryotic fungi and protists. However, in certain aspects, “higher” eukaryotic organism cells such as from insects, plants, and animals can be utilized in the methods taught herein.

Suitable host cells include, but are not limited to: bacterial cells, algal cells, plant cells, fungal cells, insect cells, and mammalian cells.

Other suitable host organisms of the present disclosure include microorganisms of the genus Corynebacterium. In some embodiments, Corynebacterium strains/species include: C. efficiens, with the deposited type strain being DSM44549, C. glutamicum, with the deposited type strain being ATCC13032, and C. ammoniagenes, with the deposited type strain being ATCC6871. In some embodiments the preferred host of the present disclosure is C. glutamicum.

Suitable host strains of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC 13870. Corynebacterium melassecola ATCC 17965, Corynebacterium thermoaminogenes FERM BP-1539, Brevibacterium flavum ATC C14067, Brevibacterium lactofermentum ATCC 13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

The term “Micrococcus glutamicus” has also been in use for C. glutamicum. Some representatives of the species C. efficiens have also been referred to as C. thermoaminogenes in the prior art, such as the strain FERM BP-1539, for example.

In some embodiments, the host cell of the present disclosure is a eukaryotic cell. Suitable eukaryotic host cells include, but are not limited to: fungal cells, algal cells, insect cells, animal cells, and plant cells. Suitable fungal host cells include, but are not limited to: Ascomycota, Basidiomycota, Deuteromycota, Zygomycota, Fungi imperfecti. Certain preferred fungal host cells include yeast cells and filamentous fungal cells. Suitable filamentous fungi host cells include, for example, any filamentous forms of the subdivision Eumycotina and Oomycota. (see, e.g., Hawksworth et al., In Ainsworth and Bisby's Dictionary of The Fungi, 8^{th} edition, 1995, CAB International, University Press, Cambridge, UK, which is incorporated herein by reference). Filamentous fungi are characterized by a vegetative mycelium with a cell wall composed of chitin, cellulose and other complex polysaccharides. The filamentous fungi host cells are morphologically distinct from yeast.

In certain illustrative, but non-limiting embodiments, the filamentous fungal host cell may be a cell of a species of: Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella, or teleomorphs, or anamorphs, and synonyms or taxonomic equivalents thereof. In one embodiment, the filamentous fungus is selected from the group consisting of A. nidulans, A. oryzae, A. sojae, and Aspergilli of the A. niger Group. In an embodiment, the filamentous fungus is Aspergillus niger.

In another embodiment, specific mutants of the fungal species are used for the methods and systems provided herein. In one embodiment, specific mutants of the fungal species are used which are suitable for the high-throughput and/or automated methods and systems provided herein. Examples of such mutants can be strains that protoplast very well; strains that produce mainly or, more preferably, only protoplasts with a single nucleus; strains that regenerate efficiently in microliter plates, strains that regenerate faster and/or strains that take up polynucleotide (e.g., DNA) molecules efficiently, strains that produce cultures of low viscosity such as, for example, cells that produce hyphae in culture that are not so entangled as to prevent isolation of single clones and/or raise the viscosity of the culture, strains that have reduced random integration (e.g., disabled non-homologous end joining pathway) or combinations thereof.

In yet another embodiment, a specific mutant strain for use in the methods and systems provided herein can be strains lacking a selectable marker gene such as, for example, uridine-requiring mutant strains. These mutant strains can be either deficient in orotidine 5 phosphate decarboxylase (OMPD) or orotate p-ribosyl transferase (OPRT) encoded by the pyrG or pyrE gene, respectively (T. Goosen et al., Curr Genet. 1987, 11:499 503; J. Begueret et al., Gene. 1984 32:487 92.

In one embodiment, specific mutant strains for use in the methods and systems provided herein are strains that possess a compact cellular morphology characterized by shorter hyphae and a more yeast-like appearance.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia (e.g. E. coli), Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable in the methods and compositions described herein.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. lichenformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure is also suitable for use with a variety of animal cell types, including mammalian cells, for example, human (including 293, WI38, PER.C6 and Bowes melanoma cells), mouse (including 3T3, NS0, NS1, Sp2/0), hamster (CHO, BHK), monkey (COS, FRhL, Vero), and hybridoma cell lines.

In various embodiments, strains that may be used in the practice of the disclosure including both prokaryotic and eukaryotic strains, are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

In some embodiments, the methods of the present disclosure are also applicable to multi-cellular organisms. For example, the platform could be used for improving the performance of crops. The organisms can comprise a plurality of plants such as Gramineae, Fetucoideae, Poacoideae, Agrostis, Phleum, Dactylis, Sorghum, Setaria, Zea, Oryza, Triticum, Secale, Avena, Hordeum, Saccharum, Poa, Festuca, Stenotaphrum, Cynodon, Coix, Olyreae, Phareae, Compositae or Leguminosae. For example, the plants can be corn, rice, soybean, cotton, wheat, rye, oats, barley, pea, beans, lentil, peanut, yam bean, cowpeas, velvet beans, clover, alfalfa, lupine, vetch, lotus, sweet clover, wisteria, sweet pea, sorghum, millet, sunflower, canola or the like. Similarly, the organisms can include a plurality of animals such as non-human mammals, fish, insects, or the like.

### Generating Genetic Diversity Pools for Utilization in the Genetic Design & HTP Microbial Engineering Platform

In some embodiments, the methods of the present disclosure are characterized as genetic design. As used herein, the term genetic design refers to the reconstruction or alteration of a host organism's genome through the identification and selection of the most optimum variants of a particular gene, portion of a gene, promoter, stop codon, 5′UTR, 3′UTR, or other DNA sequence to design and create new superior host cells.

In some embodiments, a first step in the genetic design methods of the present disclosure is to obtain an initial genetic diversity pool population with a plurality of sequence variations from which a new host genome may be reconstructed.

In some embodiments, a subsequent step in the genetic design methods taught herein is to use one or more HTP molecular tool sets (e.g., SNP swapping or promoter swapping) to construct HTP genetic design libraries, which then function as drivers of the genomic engineering process, by providing libraries of particular genomic alterations for testing in a host cell, see U.S. Pat. No. 9,988,624, issued on Jun. 5, 2018, entitled: “Microbial Strain Improvement by a HTP Genomic Engineering Platform,” incorporated by reference herein.

Harnessing Diversity Pools from Existing Wild-Type Strains

In some embodiments, the present disclosure teaches methods for identifying the sequence diversity present among microbes of a given wild-type population. Therefore, a diversity pool can be a given number n of wild-type microbes utilized for analysis, with said microbes' genomes representing the “diversity pool.”

In some embodiments, the diversity pools can be the result of existing diversity present in the natural genetic variation among said wild-type microbes. This variation may result from strain variants of a given host cell or may be the result of the microbes being different species entirely. Genetic variations can include any differences in the genetic sequence of the strains, whether naturally occurring or not. In some embodiments, genetic variations can include SNPs swaps, PRO swaps, Start/Stop Codon swaps, or STOP swaps, among others. see U.S. Pat. No. 9,988,624, issued on Jun. 5, 2018, entitled: “Microbial Strain Improvement by a HTP Genomic Engineering Platform,” incorporated by reference herein.

Harnessing Diversity Pools from Existing Strain Variants

In other embodiments of the present disclosure, diversity pools are strain variants created during traditional strain improvement processes (e.g., one or more host organism strains generated via random mutation and selected for improved yields over the years). Thus, in some embodiments, the diversity pool or host organisms can comprise a collection of historical production strains.

In particular aspects, a diversity pool may be an original parent microbial strain (S₁) with a “baseline” genetic sequence at a particular time point (S₁Gen₁) and then any number of subsequent offspring strains (S₂, S₃, S₄, S₅, etc., generalizable to S₂₋ₙ) that were derived/developed from said S₁ strain and that have a different genome (S₂₋ₙGen₂₋ₙ), in relation to the baseline genome of S₁.

For example, in some embodiments, the present disclosure teaches sequencing the microbial genomes in a diversity pool to identify the SNP's present in each strain. In one embodiment, the strains of the diversity pool are historical microbial production strains. Thus, a diversity pool of the present disclosure can include for example, an industrial base strain, and one or more mutated industrial strains produced via traditional strain improvement programs.

Once all SNPs in the diversity pool are identified, the present disclosure teaches methods of SNP swapping and screening methods to delineate (i.e. quantify and characterize) the effects (e.g. creation of a phenotype of interest) of SNPs individually and in groups. Thus, as aforementioned, an initial step in the taught platform can be to obtain an initial genetic diversity pool population with a plurality of sequence variations, e.g. SNPs. Then, a subsequent step in the taught platform can be to use one or more of the aforementioned HTP molecular tool sets (e.g. SNP swapping) to construct HTP genetic design libraries, which then function as drivers of the genomic engineering process, by providing libraries of particular genomic alterations for testing in a microbe.

In some embodiments, the SNP swapping methods of the present disclosure comprise the step of introducing one or more SNPs identified in a mutated strain (e.g., a strain from amongst S₂₋ₙGen₂₋ₙ) to a base strain (S₁Gen₁) or wild-type strain.

In other embodiments, the SNP swapping methods of the present disclosure comprise the step of removing one or more SNPs identified in a mutated strain (e.g., a strain from amongst S₂₋ₙGen₂₋ₙ). see U.S. Pat. No. 9,988,624, issued on Jun. 5, 2018, entitled: “Microbial Strain Improvement by a HTP Genomic Engineering Platform,” incorporated by reference herein.

### Creating Diversity Pools Via Mutagenesis

In some embodiments, the mutations of interest in a given diversity pool population of cells can be artificially generated by any means for mutating strains, including mutagenic chemicals, or radiation. The term “mutagenizing” is used herein to refer to a method for inducing one or more genetic modifications in cellular nucleic acid material.

The term “genetic modification” refers to any alteration of DNA. Representative gene modifications include nucleotide insertions, deletions, substitutions, and combinations thereof, and can be as small as a single base or as large as tens of thousands of bases. Thus, the term “genetic modification” encompasses inversions of a nucleotide sequence and other chromosomal rearrangements, whereby the position or orientation of DNA comprising a region of a chromosome is altered. A chromosomal rearrangement can comprise an intrachromosomal rearrangement or an interchromosomal rearrangement.

In one embodiment, the mutagenizing methods employed in the presently claimed subject matter are substantially random such that a genetic modification can occur at any available nucleotide position within the nucleic acid material to be mutagenized. Stated another way, in one embodiment, the mutagenizing does not show a preference or increased frequency of occurrence at particular nucleotide sequences.

The methods of the disclosure can employ any mutagenic agent including, but not limited to: ultraviolet light, X-ray radiation, gamma radiation, N-ethyl-N-nitrosourea (ENU), methyinitrosourea (MNU), procarbazine, (PRC), triethylene melamine (TEM), acrylamide monomer (AA), chlorambucil (CHL), melphalan (MLP), cyclophosphamide (CPP), diethyl sulfate (DES), ethyl methane sulfonate (EMS), methyl methane sulfonate (MMS), 6-mercaptopurine (6-MP), mitomycin-C (MMC), N-methyl-N′-nitro-N-nitrosoguanidine (MNNG), ‘H₂O, and urethane (UR) (See e.g., Rinchik, 1991 Marker et al., 1997; and Russell, 1990). Additional mutagenic agents are well known to persons having skill in the art, including those described in http://www.iephb.nw.ru/˜spirov/hazard/mutagen_1st.html.

The term “mutagenizing” also encompasses a method for altering (e.g., by targeted mutation) or modulating a cell function, to thereby enhance a rate, quality, or extent of mutagenesis. For example, a cell can be altered or modulated to thereby be dysfunctional or deficient in DNA repair, mutagen metabolism, mutagen sensitivity, genomic stability, or combinations thereof. Thus, disruption of gene functions that normally maintain genomic stability can be used to enhance mutagenesis. Representative targets of disruption include, but are not limited to DNA ligase I (Bentley et al., 2002) and casein kinase I (U.S. Pat. No. 6,060,296).

In some embodiments, site-specific mutagenesis (e.g., primer-directed mutagenesis using a commercially available kit such as the Transformer Site Directed mutagenesis kit (Clontech)) is used to make a plurality of changes throughout a nucleic acid sequence in order to generate nucleic acid encoding a cleavage enzyme of the present disclosure.

The frequency of genetic modification upon exposure to one or more mutagenic agents can be modulated by varying dose and/or repetition of treatment, and can be tailored for a particular application.

Thus, in some embodiments, “mutagenesis” as used herein comprises all techniques known in the art for inducing mutations, including error-prone PCR mutagenesis, oligonucleotide-directed mutagenesis, site-directed mutagenesis, and iterative sequence recombination by any of the techniques described herein.

### Single Locus Mutations to Generate Diversity and Targeted Genetic Editing of Host Cells

In some embodiments, the present disclosure teaches mutating cell populations by introducing, deleting, or replacing selected portions of genomic DNA. Thus, in some embodiments, the present disclosure teaches methods for targeting mutations to a specific locus. In other embodiments, the present disclosure teaches the use of gene editing technologies such as ZFNs, TALENS, or CRISPR (utilizing any RNA guided nuclease, e.g. Cas9, Cpf1, etc.), to selectively edit target DNA regions.

In other embodiments, the present disclosure teaches mutating selected DNA regions outside of the host organism, and then inserting the mutated sequence back into the host organism. For example, in some embodiments, the present disclosure teaches mutating native or synthetic promoters to produce a range of promoter variants with various expression properties (see promoter ladder infra). In other embodiments, the present disclosure is compatible with single gene optimization techniques, such as ProSAR (Fox et al. 2007. “Improving catalytic function by ProSAR-driven enzyme evolution,” Nature Biotechnology Vol 25 (3) 338-343, incorporated by reference herein).

In some embodiments, the selected regions of DNA are produced in vitro via gene shuffling of natural variants, or shuffling with synthetic oligos, plasmid-plasmid recombination, virus plasmid recombination, virus-virus recombination. In other embodiments, the genomic regions are produced via error-prone PCR (see e.g., FIG. 1).

In some embodiments, generating mutations in selected genetic regions is accomplished by “reassembly PCR.” Briefly, oligonucleotide primers (oligos) are synthesized for PCR amplification of segments of a nucleic acid sequence of interest, such that the sequences of the oligonucleotides overlap the junctions of two segments. The overlap region is typically about 10 to 100 nucleotides in length. Each of the segments is amplified with a set of such primers. The PCR products are then “reassembled” according to assembly protocols. In brief, in an assembly protocol, the PCR products are first purified away from the primers, by, for example, gel electrophoresis or size exclusion chromatography. Purified products are mixed together and subjected to about 1-10 cycles of denaturing, reannealing, and extension in the presence of polymerase and deoxynucleoside triphosphates (dNTP's) and appropriate buffer salts in the absence of additional primers (“self-priming”). Subsequent PCR with primers flanking the gene are used to amplify the yield of the fully reassembled and shuffled genes.

In some embodiments of the disclosure, mutated DNA regions, such as those discussed above, are enriched for mutant sequences so that the multiple mutant spectrum, i.e. possible combinations of mutations, is more efficiently sampled. In some embodiments, mutated sequences are identified via a mutS protein affinity matrix (Wagner et al., Nucleic Acids Res. 23(19):3944-3948 (1995); Su et al., Proc. Natl. Acad. Sci. (U.S.A.), 83:5057-5061(1986)) with a preferred step of amplifying the affinity-purified material in vitro prior to an assembly reaction. This amplified material is then put into an assembly or reassembly PCR reaction as described in later portions of this application.

### HTP Robotic Systems

In some embodiments, the automated methods of the disclosure comprise a robotic system. The systems outlined herein are generally directed to the use of 96-well and 6-well microliter plates, but as will be appreciated by those in the art, any number of different plates or configurations may be used. In addition, any or all of the steps outlined herein may be automated; thus, for example, the systems may be completely or partially automated.

In some embodiments, the automated systems of the present disclosure comprise one or more work modules. For example, in some embodiments, the automated system of the present disclosure comprises a DNA synthesis module, a vector cloning module, a strain transformation module, a screening module, and a sequencing module.

As will be appreciated by those in the art, an automated system can include a wide variety of components, including, but not limited to: liquid handlers; one or more robotic arms; plate handlers for the positioning of microplates; plate sealers, plate piercers, automated lid handlers to remove and replace lids for wells on non-cross contamination plates; disposable tip assemblies for sample distribution with disposable tips; washable tip assemblies for sample distribution; 96 well loading blocks; integrated thermal cyclers; cooled reagent racks; microtiter plate pipette positions (optionally cooled); stacking towers for plates and tips; magnetic bead processing stations; filtrations systems; plate shakers; barcode readers and applicators; and computer systems.

In some embodiments, the robotic systems of the present disclosure include automated liquid and particle handling enabling high-throughput pipetting to perform all the steps in the process of gene targeting and recombination applications. This includes liquid and particle manipulations such as aspiration, dispensing, mixing, diluting, washing, accurate volumetric transfers; retrieving and discarding of pipette tips; and repetitive pipetting of identical volumes for multiple deliveries from a single sample aspiration. These manipulations are cross-contamination-free liquid, particle, cell, and organism transfers. The instruments perform automated replication of microplate samples to filters, membranes, and/or daughter plates, high-density transfers, full-plate serial dilutions, and high capacity operation.

In some embodiments, the customized automated liquid handling system of the disclosure is a TECAN machine (e.g. a customized TECAN Freedom Evo).

In some embodiments, the automated systems of the present disclosure are compatible with platforms for multi-well plates, deep-well plates, square well plates, reagent troughs, test tubes, mini tubes, microfuge tubes, cryovials, filters, micro array chips, optic fibers, beads, agarose and acrylamide gels, and other solid-phase matrices or platforms are accommodated on an upgradeable modular deck. In some embodiments, the automated systems of the present disclosure contain at least one modular deck for multi-position work surfaces for placing source and output samples, reagents, sample and reagent dilution, assay plates, sample and reagent reservoirs, pipette tips, and an active tip-washing station.

In some embodiments, the automated systems of the present disclosure include high-throughput electroporation systems. In some embodiments, the high-throughput electroporation systems are capable of transforming cells in 96 or 384-well plates. In some embodiments, the high-throughput electroporation systems include VWR® High-throughput Electroporation Systems, BTX™, Bio-Rad® Gene Pulser MXcell™ or other multi-well electroporation system.

In some embodiments, the integrated thermal cycler and/or thermal regulators are used for stabilizing the temperature of heat exchangers such as controlled blocks or platforms to provide accurate temperature control of incubating samples from 0° C. to 100° C.

In some embodiments, the automated systems of the present disclosure are compatible with interchangeable machine-heads (single or multi-channel) with single or multiple magnetic probes, affinity probes, replicators or pipetters, capable of robotically manipulating liquid, particles, cells, and multi-cellular organisms. Multi-well or multi-tube magnetic separators and filtration stations manipulate liquid, particles, cells, and organisms in single or multiple sample formats.

In some embodiments, the automated systems of the present disclosure are compatible with camera vision and/or spectrometer systems. Thus, in some embodiments, the automated systems of the present disclosure are capable of detecting and logging color and absorption changes in ongoing cellular cultures.

In some embodiments, the automated system of the present disclosure is designed to be flexible and adaptable with multiple hardware add-ons to allow the system to carry out multiple applications. The software program modules allow creation, modification, and running of methods. The system's diagnostic modules allow setup, instrument alignment, and motor operations. The customized tools, labware, and liquid and particle transfer patterns allow different applications to be programmed and performed. The database allows method and parameter storage. Robotic and computer interfaces allow communication between instruments.

Thus, in some embodiments, the present disclosure teaches a high-throughput strain engineering platform.

Persons having skill in the art will recognize the various robotic platforms capable of carrying out the FITP engineering methods of the present disclosure. Table 1 below provides a non-exclusive list of scientific equipment capable of carrying out each step of the HTP steps of the present disclosure.

TABLE 1
Non-exclusive list of Scientific Equipment Compatible
with the HTP engineering methods of the disclosure

| | Equipment | | Compatible Equipment |
|---|---|---|---|
| | Type | Operation(s) performed | Make/Model/Configuration |
| Acquire and build | liquid handlers | Hitpicking (combining by | Hamilton Microlab STAR, |
| DNA pieces | | transferring) | Labcyte Echo 550, Tecan EVO |
| | | primers/templates for PCR | 200, Beckman Coulter Biomek |
| | | amplification of DNA | FX, or equivalents |
| | | parts | |
| | Thermal cyclers | PCR amplification of | Inheco Cycler, ABI 2720, ABI |
| | | DNA parts | Proflex 384, ABI Veriti, or |
| | | | equivalents |
| QC DNA parts | Fragment | gel electrophoresis to | Agilent Bioanalyzer, AATI |
| | analyzers | confirm PCR products of | Fragment Analyzer, or |
| | (capillary | appropriate size | equivalents |
| | electrophoresis) | | |
| | Sequencer | Verifying sequence of | Beckman Ceq-8000, Beckman |
| | (sanger: | parts/templates | GenomeLab ™, or equivalents |
| | Beckman) | | |
| | NGS (next | Verifying sequence of | Illumina MiSeq series |
| | generation | parts/templates | sequences, illumina Hi-Seq, Ion |
| | sequencing) | assessing concentration of | torrent, pac bio or other |
| | instrument | DNA samples | equivalents |
| | nanodrop/plate | | Molecular Devices SpectraMax |
| | reader | | M5, Tecan Ml000, or |
| | | | equivalents. |
| Generate DNA | liquid handlers | Hitpicking (combining by | Hamilton Microlab STAR, |
| assembly | | transferring) DNA parts | Labcyte Echo 550, Tecan EVO |
| | | for assembly along with | 200, Beckman Coulter Biomek |
| | | cloning vector, addition of | FX, or equivalents |
| | | reagents for assembly | |
| | | reaction/process | |
| QC DNA | Colony pickers | for inoculating colonies in | Scirobotics Pickolo, Molecular |
| assembly | | liquid media | Devices QPix 420 |
| | liquid handlers | Hitpicking | Hamilton Microlab STAR, |
| | | primers/templates, diluting | Labcyte Echo 550, Tecan EVO |
| | | samples | 200, Beckman Coulter Biomek |
| | | | FX, or equivalents |
| | Fragment | gel electrophoresis to | Agilent Bioanalyzer, AATI |
| | analyzers | confirm assembled | Fragment Analyzer |
| | (capillary | products of appropriate | |
| | electrophoresis) | size | |
| | Sequencer | Verifying sequence of | ABI3730 Thermo Fisher, |
| | (sanger: | assembled plasmids | Beckman Ceq-8000, Beckman |
| | Beckman) | | GenomeLab ™, or equivalents |
| | NGS (next | Verifying sequence of | Illumina MiSeq series |
| | generation | assembled plasmids | sequences, illumina Hi-Seq, Ion |
| | sequencing) | | torrent, pac bio or other |
| | instrument | | equivalents |
| Prepare base strain | centrifuge | spinning/pelleting cells | Beckman Avanti floor |
| and DNA assembly | | | centrifuge, |
| | | | Hettich Centrifuge |
| Transform DNA | Electroporators | electroporative | BTX Gemini X2, BIO-RAD |
| into base strain | | transformation of cells | MicroPulser Electroporator |
| | Ballistic | ballistic transformation of | BIO-RAD PDS1000 |
| | transformation | cells | |
| | Incubators, | for chemical | Inheco Cycler, ABI 2720, ABI |
| | thermal cyclers | transformation/heat shock | Proflex 384, ABI Veriti, or |
| | | | equivalents |
| | Liquid handlers | for combining DNA, cells, | Hamilton Microlab STAR, |
| | | buffer | Labcyte Echo 550, Tecan EVO |
| | | | 200, Beckman Coulter Biomek |
| | | | FX, or equivalents |
| Integrate DNA into | Colony pickers | for inoculating colonies in | Scirobotics Pickolo, Molecular |
| genome of base strain | | liquid media | Devices QPix 420 |
| | Liquid handlers | For transferring cells onto | Hamilton Microlab STAR, |
| | | Agar, transferring from | Labcyte Echo 550, Tecan EVO |
| | | culture plates to different | 200, Beckman Coulter Biomek |
| | | culture plates (inoculation | FX, or equivalents |
| | | into other selective media) | |
| | Platform | incubation with shaking of | Kuhner Shaker ISF4-X, Infors- |
| | shaker- | microtiter plate cultures | ht Multitron Pro |
| | incubators | | |
| QC transformed | Colony pickers | for inoculating colonies in | Scirobotics Pickolo, Molecular |
| strain | | liquid media | Devices QPix 420 |
| | liquid handlers | Hitpicking | Hamilton Microlab STAR, |
| | | primers/templates, diluting | Labcyte Echo 550, Tecan EVO |
| | | samples | 200, Beckman Coulter Biomek |
| | | | FX, or equivalents |
| | Thermal cyclers | cPCR verification of | Inheco Cycler, ABI 2720, ABI |
| | | strains | Proflex 384, ABI Veriti, or |
| | | | equivalents |
| | Fragment | gel electrophoresis to | Infors-ht Multitron Pro, Kuhner |
| | analyzers | confirm cPCR products of | Shaker ISF4-X |
| | (capillary | appropriate size | |
| | electrophoresis) | | |
| | Sequencer | Sequence verification of | Beckman Ceq-8000, Beckman |
| | (sanger: | introduced modification | GenomeLab ™, or equivalents |
| | Beckman) | | |
| | NGS (next | Sequence verification of | Illumina MiSeq series |
| | generation | introduced modification | sequences, illumina Hi-Seq, Ion |
| | sequencing) | | torrent, pac bio or other |
| | instrument | | equivalents |
| Select and | Liquid handlers | For transferring from | Hamilton Microlab STAR, |
| consolidate | | culture plates to different | Labcyte Echo 550, Tecan EVO |
| QC'd strains | | culture plates (inoculation | 200, Beckman Coulter Biomek |
| into test plate | | into production media) | FX, or equivalents |
| | Colony pickers | for inoculating colonies in | Scirobotics Pickolo, Molecular |
| | | liquid media | Devices QPix 420 |
| | Platform | incubation with shaking of | Kuhner Shaker ISF4-X, Infors- |
| | shaker- | microtiter plate cultures | ht Multitron Pro |
| | incubators | | |
| Culture strains | Liquid handlers | For transferring from | Hamilton Microlab STAR, |
| in seed plates | | culture plates to different | Labcyte Echo 550, Tecan EVO |
| | | culture plates (inoculation | 200, Beckman Coulter Biomek |
| | | into production media) | FX, or equivalents |
| | Platform | incubation with shaking of | Kuhner Shaker ISF4-X, Infors- |
| | shaker- | microtiter plate cultures | ht Multitron Pro |
| | incubators | | |
| | liquid | Dispense liquid culture | Well mate (Thermo), |
| | dispensers | media into microtiter | Benchcel2R (velocity 11), |
| | | plates | plateloc (velocity 11) |
| | microplate | apply barcoders to plates | Microplate labeler (a2+ cab - |
| | labeler | | agilent), benchcell 6R |
| | | | (velocity 11) |
| Generate product | Liquid handlers | For transferring from | Hamilton Microlab STAR, |
| from strain | | culture plates to different | Labcyte Echo 550, Tecan EVO |
| | | culture plates (inoculation | 200, Beckman Coulter Biomek |
| | | into production media) | FX, or equivalents |
| | Platform | incubation with shaking of | Kuhner Shaker ISF4-X, Infors- |
| | shaker- | microtiter plate cultures | ht Multitron Pro |
| | incubators | | |
| | liquid | Dispense liquid culture | well mate (Thermo), |
| | dispensers | media into multiple | Benchcel2R (velocity 11), |
| | | microtiter plates and seal | plateloc (velocity 11) |
| | | plates | |
| | microplate | Apply barcodes to plates | microplate labeler (a2+ cab - |
| | labeler | | agilent), benchcell 6R |
| | | | (velocity 11) |
| Evaluate | Liquid handlers | For processing culture | Hamilton Microlab STAR, |
| performance | | broth for downstream | Labcyte Echo 550, Tecan EVO |
| | | analytical | 200, Beckman Coulter Biomek |
| | | | FX, or equivalents |
| | UHPLC, HPLC | quantitative analysis of | Agilent 1290 Series UHPLC |
| | | precursor and target | and 1200 Series HPLC with |
| | | compounds | UV and RI detectors, or |
| | | | equivalent; also any LC/MS |
| | LC/MS | highly specific analysis of | Agilent 6490 QQQ and 6550 |
| | | precursor and target | QTOF coupled to 1290 Series |
| | | compounds as well as side | UHPLC |
| | | and degradation products | |
| | Spectrophotometer | Quantification of different | Tecan M1000, spectramax M5, |
| | | compounds using | Genesys 10S |
| | | spectrophotometer based | |
| | | assays | |
| Culture strains | Fermenters: | incubation with shaking | Sartorius, DASGIPs |
| in flasks | | | (Eppendorf), BIO-FLOs |
| | | | (Sartorius-stedim). Applikon |
| | Platform | | innova 4900, or any equivalent |
| | shakers | | |
| Generate product | Fermenters: DASGIPs (Eppendorf), BIO-FLOs (Sartorius-stedim) | | |
| from strain | | | |
| Evaluate | Liquid handlers | For transferring from | Hamilton Microlab STAR, |
| performance | | culture plates to different | Labcyte Echo 550, Tecan EVO |
| | | culture plates (inoculation | 200, Beckman Coulter Biomek |
| | | into production media) | FX, or equivalents |
| | UHPLC, HPLC | quantitative analysis of | Agilent 1290 Series UHPLC |
| | | precursor and target | and 1200 Series HPLC with |
| | | compounds | UV and RI detectors, or |
| | | | equivalent; also any LC/MS |
| | LC/MS | highly specific analysis of | Agilent 6490 QQQ and 6550 |
| | | precursor and target | QTOF coupled to 1290 Series |
| | | compounds as well as side | UHPLC |
| | | and degradation products | |
| | Flow cytometer | Characterize strain | BD Accuri, Millipore Guava |
| | | performance (measure | |
| | | viability) | |
| | Spectrophotometer | Characterize strain | Tecan M1000, Spectramax M5, |
| | | performance (measure | or other equivalents |
| | | biomass) | |

### Computer System Hardware

A computer system or compute device (e.g., compute device 102 of FIG. 1) of the present disclosure may be used to execute program code stored in a non-transitory computer readable medium (e.g., memory), in accordance with embodiments of the disclosure. The computer system can includes an input/output subsystem, which may be used to interface with human users and/or other computer systems depending upon the application. The I/O subsystem may include, e.g., a keyboard, mouse, graphical user interface, touchscreen, or other interfaces for input, and, e.g., an LED or other flat screen display, or other interfaces for output, including application program interfaces (APIs).

Program code may be stored in non-transitory media such as persistent storage in secondary memory, main memory, or both. Main memory may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors read program code from one or more non-transitory media and executes the code to enable the computer system to accomplish the methods performed by the embodiments herein. Those skilled in the art will understand that the processor(s) may ingest source code, and interpret or compile the source code into machine code that is understandable at the hardware gate level of the processor(s). The processor(s) may include graphics processing units (GPUs) for handling computationally intensive tasks. Particularly in machine learning, one or more CPUs may offload the processing of large quantities of data to one or more GPUs.

The processor(s) may communicate with external networks via one or more communications interfaces, such as a network interlace card, WiFi transceiver, etc. A bus communicatively couples the I/O subsystem, the processor(s), peripheral devices, communications interfaces, memory, and persistent storage. Embodiments of the disclosure are not limited to this representative architecture. Alternative embodiments may employ different arrangements and types of components, e.g., separate buses for input-output components and memory subsystems.

Those skilled in the art will understand that some or all of the elements of embodiments of the disclosure, and their accompanying operations, may be implemented wholly or partially by one or more computer systems including one or more processors and one or more memory systems. In particular, any robotics and other automated systems or devices described herein may be computer-implemented. Some elements and functionality may be implemented locally and others may be implemented in a distributed fashion over a network through different servers, e.g., in client-server fashion, for example. In particular, server-side operations may be made available to multiple clients in a software as a service (SaaS) fashion.

The term component in this context refers broadly to software, hardware, or firmware (or any combination thereof) component. Components are typically functional components that can generate useful data or other output using specified input(s). A component may or may not be self-contained. An application program (also called an “application”) may include one or more components, or a component can include one or more application programs.

Some embodiments include some, all, or none of the components along with other modules or application components. Still yet, various embodiments may incorporate two or more of these components into a single module and/or associate a portion of the functionality of one or more of these components with a different component.

The term “memory” can be any device or mechanism used for storing information. In accordance with some embodiments of the present disclosure, memory is intended to encompass any type of, but is not limited to: volatile memory, nonvolatile memory, and dynamic memory. For example, memory can be random access memory, memory storage devices, optical memory devices, magnetic media, floppy disks, magnetic tapes, hard drives, SIMMs, SDRAM, DIMMs, RDRAM, DDR RAM, SODIMMS, erasable programmable read-only memories (EPROMs), electrically erasable programmable read-only memories (EEPROMs), compact disks, DVDs, and/or the like. In accordance with some embodiments, memory may include one or more disk drives, flash drives, databases, local cache memories, processor cache memories, relational databases, flat databases, servers, cloud based platforms, and/or the like. In addition, those of ordinary skill in the art will appreciate many additional devices and techniques for storing information can be used as memory.

Memory may be used to store instructions for running one or more applications or modules on a processor. For example, memory could be used in some embodiments to house all or some of the instructions needed to execute the functionality of one or more of the modules and/or applications disclosed in this application.

FIG. 2 is a flow diagram showing a method for automated, high throughput cellular library generation, according to some embodiments. The method 200 of FIG. 2 can be performed, for example, using the system 100 of FIG. 1. As shown in FIG. 2, the method 200 includes providing a suspension including transformed cells, at 212, and at 216, plating the transformed cells onto solid surfaces of each of at least one reservoir of a reservoir plate. The solid surfaces of each of at least one reservoir of a reservoir plate can include a liquid growth medium. The reservoir plate is incubated, at 218, and after cellular growth has occurred on at least one plated surface of the reservoir plate, a series of steps (steps 220 through 224, and optionally steps 226 and/or 228) are performed automatically. The automatically-performed steps include one or more of adding disaggregation solution to the reservoir plate (220), applying a mechanical force to the reservoir plate to produce resuspended cells (222), collecting the resuspended cells (224), processing the collected resuspended cells (226), and preserving the collected resuspended cells (228). The disaggregation solution added at 220 can include art least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include a viscous liquid. Alternatively or in addition, the disaggregation solution includes at least one of a positive selection agent or a negative selection agent. Alternatively or in addition, the disaggregation solution includes a microbial growth medium, glycerol, and an antibiotic. The mechanical force applied at 222 can be, for example, a rotational force between 200 RPM and 3,000 RPM. The mechanical force can be applied for a duration, for example, of between 10 seconds and 10 minutes, or between 30 seconds and 60 seconds. The automatically-performed steps can also include transferring the collected resuspended cells to a multi-well plate after the cellular growth has occurred on at least one plated surface of the reservoir plate. The method 200 optionally also includes, at 210, introducing DNA to cells to produce the transformed cells, prior to the plating at 216. Alternatively or in addition, the method 200 can also include, at 214, waiting a predetermined recovery duration after producing the transformed cells and prior to the plating at 216. The at least one reservoir of the reservoir plate can include, for example, a relatively small number of reservoirs (also referred to herein as “wells”), for example 1 reservoir or 2 reservoirs (e.g., if a larger surface is needed, such as may be the case for mammalian cells), or a larger number of reservoirs, for example 6 reservoirs, 12 reservoirs, 24 reservoirs, 96 reservoirs, or 384 reservoirs.

In some embodiments, for example when a liquid growth medium is used, the reservoir plate is not prepared in advance of the plating. In other embodiments, the reservoir plate is a prepared prior to the plating, for example by applying a predetermined volume of a growth medium into each well of the reservoir plate.

In some embodiments, the method 100 includes preserving the collected resuspended cells (228) at a predetermined temperature, for example at −80° C., or at −130° C. (e.g., mammalian cells), or at −20° C. (e.g., for applications in which cell viability is not a priority), or between −130° C. and −20° C., or between −80° C. and −20° C., or between −130° C. and −80° C.

In some embodiments, after the plating at 216 of the transformed cells, each reservoir from the reservoir plate includes a different associated plasmid.

In some embodiments, after the cellular growth has occurred on the at least one plated surface of the reservoir plate, and prior to applying the mechanical force (at 222) to the reservoir plate, beads are added to the reservoir plate. The beads can comprise, for example, glass or stainless steel, and can have a diameter on the order of millimeters (e.g., 1 mm to 5 mm) or larger. Example beads compatible with systems set forth herein are Rattler Plating Beads (e.g., S1001, S1001-5 or S1001-B) available from Zymo Research.

In some embodiments, the method 200 also includes controlling a temperature of the reservoir plate during the application of the mechanical force, such that the temperature of the reservoir plate is between 4° C. and 40° C.

FIG. 3 is a flow diagram showing a method of removing a plurality of discrete colonies from solid supports of a reservoir plate, according to some embodiments. The method 300 of FIG. 3 can be performed, for example, using the system 100 of FIG. 1. As shown in FIG. 3, the method 300 includes controlling a robotic arm, at 330, to cause transfer of a suspension including a population of cells to each of a plurality of reservoirs of the reservoir plate. The method 300 also includes detecting, at 332 and after transferring the suspension to the reservoir plate, colony growth on solid supports of reservoirs of the reservoir plate. For example, step 332 of method 300 can include detecting that each discrete colony from the plurality or set of discrete colonies has grown on an associated solid support of an associated reservoir from the plurality of reservoirs of the reservoir plate. In response to detecting the cellular growth, at 334, a disaggregation solution is introduced to the reservoir plate. The disaggregation solution can include a viscous liquid. Alternatively or in addition, the disaggregation solution can include at least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include at least one of a positive selection agent or a negative selection agent Alternatively or in addition, the disaggregation solution can include a microbial growth medium, glycerol, and an antibiotic. A mechanical force is applied at 336 to the reservoir plate to produce resuspended cells within each reservoir from the plurality of reservoirs of the reservoir plate. The mechanical force can include a. rotational force between 200 RPM and 3,000 RPM, and/or can be applied for a predetermined duration (e.g., between 10 seconds and 10 minutes, or between 30 seconds and 60 seconds). The method 300 optionally also includes performing an automated library preparation, at 338, using the resuspended cells. The method 300 optionally also includes adding beads to the reservoir plate prior to applying the mechanical force to the reservoir plate.

In some embodiments, the method 300 also includes controlling a temperature of the reservoir plate during the application of the mechanical force at 336, such that the temperature of the reservoir plate is maintained at between 4° C. and 40° C.

In some embodiments, a method includes providing a suspension including a population of cells (e.g., transformed cells), plating cells from the population of cells onto solid surfaces of each of at least one reservoir of a reservoir plate, and incubating the reservoir plate. After cellular growth has occurred on at least one plated surface of the reservoir plate, the method also includes automatically adding disaggregation solution to the reservoir plate, automatically applying a mechanical force to the reservoir plate to produce resuspended cells, and automatically collecting the resuspended cells. The disaggregation solution can include a viscous liquid. Alternatively or in addition, the disaggregation solution can include at least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include at least one of a positive selection agent or a negative selection agent. Alternatively or in addition, the disaggregation solution can include a microbial growth medium, glycerol, and an antibiotic. The mechanical force can include a rotational force between 200 RPM and 3,000 RPM, and/or can be applied for a predetermined duration (e.g., between 10 seconds and 10 minutes, or between 30 seconds and 60 seconds).

In some embodiments, the population of cells includes culturable bacteria. In other embodiments, the population of cells includes at least one of: edited microbes, or natural microbes.

In some embodiments, the method also includes automatically transferring the collected resuspended cells to a multi-well plate after the cellular growth has occurred on at least one plated surface of the reservoir plate.

In some embodiments, the method also includes performing an automated library preparation using the collected resuspended cells.

In some embodiments, the method also includes adding beads to the reservoir plate prior to applying the mechanical force to the reservoir plate.

In some embodiments, the method also includes controlling a temperature of the reservoir plate during the application of the mechanical force, such that the temperature of the reservoir plate is between 4° C. and 40° C.

In some embodiments, a method includes providing a suspension including a population of transfected cells (e.g., mammalian cells), transferring the population of transfected cells to at least one reservoir of a reservoir plate, and incubating the reservoir plate. After cellular growth has occurred within the reservoir plate, the method further includes automatically adding disaggregation solution to the reservoir plate, automatically applying a mechanical force to the reservoir plate to produce resuspended cells, and automatically collecting the resuspended cells.

The disaggregation solution can include a viscous liquid. Alternatively or in addition, the disaggregation solution can include at least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include at least one of a positive selection agent or a negative selection agent. Alternatively or in addition, the disaggregation solution can include a microbial growth medium, glycerol, and an antibiotic. The mechanical force can include a rotational force between 200 RPM and 3,000 RPM, and/or can be applied for a predetermined duration (e.g., between 10 seconds and 10 minutes, or between 30 seconds and 60 seconds).

In some embodiments, the method also includes performing an automated library preparation based on the collected resuspended cells.

In some embodiments, the method also includes adding beads to the reservoir plate prior to applying the mechanical force to the reservoir plate.

In some embodiments, the method also includes controlling a temperature of the reservoir plate during the application of the mechanical force, such that the temperature of the reservoir plate is between 4° C. and 40° C.

In some embodiments, a spore production and harvesting method includes providing a suspension including a population of cells, plating cells from the population of cells onto solid surfaces of each of at least one reservoir of a reservoir plate, and incubating the reservoir plate. The solid surfaces of the at least one reservoir of the reservoir plate can include, for example, an antibiotic. After cultures have sporulated on at least one plated surface of the reservoir plate, the method also includes automatically adding disaggregation solution to the reservoir plate, automatically applying a mechanical force to the reservoir plate to produce a spore suspension including at least a portion of the spores, and automatically collecting the spore suspension. The disaggregation solution can include a viscous liquid. Alternatively or in addition, the disaggregation solution can include at least one of: Triton Z100, Tween-80, Urea, Pluronic™ F-68, Accumax, Accutase, Lipase, a detergent, a protease, an amilase, a cellulose, or a glycerol. Alternatively or in addition, the disaggregation solution can include at least one of a positive selection agent or a negative selection agent. Alternatively or in addition, the disaggregation solution can include a microbial growth medium, glycerol, and an antibiotic. The mechanical force can include a rotational force between 200 RPM and 3,000 RPM, and/or can be applied for a predetermined duration (e.g., between 10 seconds and 10 minutes, or between 30 seconds and 60 seconds).

In some embodiments, the method also includes performing an automated library preparation based on the spore suspension.

In some embodiments, the method also includes adding beads to the reservoir plate prior to applying the mechanical force to the reservoir plate.

In some embodiments, the method also includes controlling a temperature of the reservoir plate during the application of the mechanical force, such that the temperature of the reservoir plate is between 4° C. and 40° C.

FIG. 4 is an overview of a library generation process, according to some embodiments. As shown in FIG. 4, the library generation process 400 includes introducing DNA into cells, at 440, to produce transformed cells (“transformation”). At 442, the transformed cells are allowed to recover, in liquid (e.g., using one or more multiple-well reservoir plates, such as a 96-well reservoir plate, in a first format), from the transformation process. At 444, the recovered transformed cells are plated onto one or more multiple-well reservoir plates, such as a 6-well reservoir plate (a second format), selectively grown, and permitted to recover, to produce a cellular library. After the selective growth at 444 has completed, cells and/or groups thereof are resuspended and placed back into first format reservoir plate, at 446, for downstream processing (e.g., quality control).

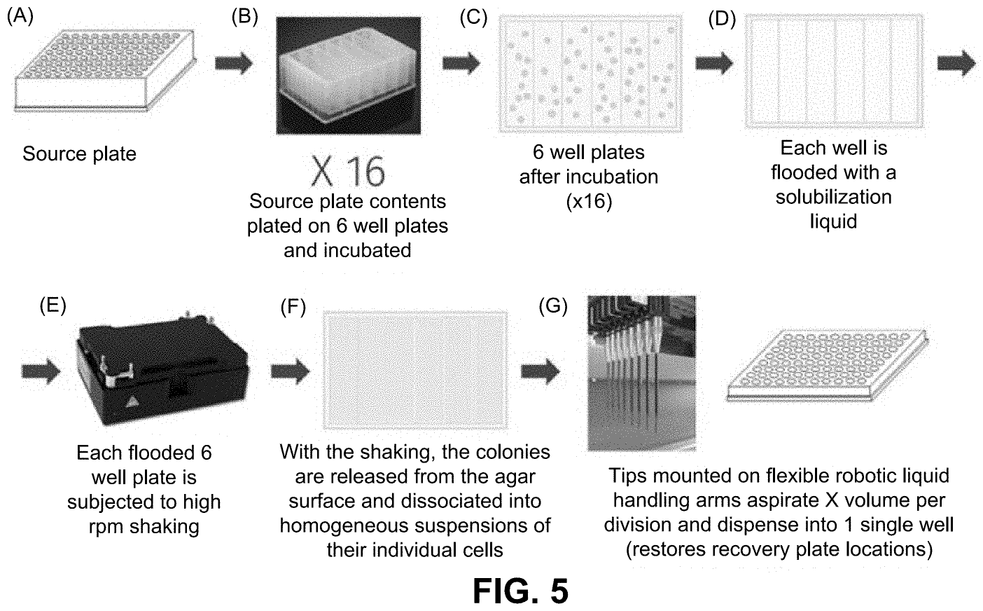

FIG. 5 is an outline of an example application of the process of FIG. 4. The steps shown in this FIG. 5 are an example implementation of step 444 of method 400 in FIG. 4. As shown in FIG. 5, one or more source reservoir plates (“source plates”) (A), for example a single 96-well source reservoir plate or multiple source reservoir plates totaling 96 wells, are provided, and the source plate contents are transferred into one or more 6-well reservoir plates (“process plates”) each having 6 deep wells with agar-containing surfaces. At (B), cells are plated onto the agar-containing surfaces of sixteen 6-well process plate(s) and incubated to produce incubated cells that have undergone colony growth, at (C). At (D), each of the 6 wells of the process plate(s) is flooded with a solubilization solution that assists the mechanical forces of a bioshaker (E). At (E), each flooded 6-well process plate is subjected to high RPM shaking. As shown at (F), the high RPM shaking causes the colonies to be released from the agar-containing surfaces. Once released from the agar-containing surfaces, the individual cells are mixed together, and the cell colonies become dissociated into homogeneous suspensions of their individual cells within the process plate(s). Once the colonies have been disrupted and the homogenous suspensions have been generated, aliquots of such suspension are collected (at G) from the process plate(s) and transferred back to the source plate(s) for downstream processing. To accomplish the transfer, tips mounted on flexible robotic liquid handling arms aspirate a predetermined volume (“X”) per well/division of the process plate(s) and dispense said volume into a single associated well of the source plate. Table 2, below, shows example material sources, material types, and labware (“format”) types for each step in the process of FIG. 5.

TABLE 2
Example Materials Used in the Process of FIG. 5

| FIG. 5 Step | (A) | (B) | (C) | (D)-(F) | (G) |
|---|---|---|---|---|---|
| Material | Transformation/ | Result of | Result of | Result of | Result of |
| source | transfection/ | step (A) | step (B), | step (C) | step (F) |
| | transduction | | post-growth | | |
| Material | Cells in | Cells in | Cells on solid | Cells in | Cells in |
| type | suspension | suspension | surface | suspension | suspension |
| Format | Multi-well, round- | Deep well | Deep well | Deep well | Multi, round- |
| | well plate (6+ | plate | plate | plate | well plate (6+ |
| | wells, e.g., 96 | (e.g., 6 wells | (e.g., 6 well | (e.g., 6 wells | wells, e.g. |
| | wells or 384 | or 12 wells) | s or 12 wells) | or 12 wells) | 96w or 384w - |
| | wells) | | | | same as step |
| | | | | | a) |

In some embodiments of the method for automated, high throughput cellular library generation, cells from a library of cells with multiple uncharacterized genotypes (e.g., different plasmids in one single well of a 96-well reservoir plate, as shown at (A) in FIG. 5) are distributed over the surface of one well of a 6-well reservoir plate (see (B) in FIG. 5) and distributed with a manual tilt of the plate or a gentle robotic-mediated spreading over the bottom of the well of the 6-well reservoir plate. The library of cells with uncharacterized genotypes grows into colonies (at (C) in FIG. 5), and these can be removed from the surface of the matrix or bottom of the well by adding a solution (at (I)) in FIG. 5) and subjecting the 6-well reservoir plate to vigorous shaking (at (E) in FIG. 5). The mechanical forces, together with the disaggregation solution, allow for the release of the groups of cells from the matrix and for the mixing of the cells from the previously separated groups of cells (at (F) in FIG. 5). The mixed, resuspended cells are then ready for collection and transfer back to the 96-well reservoir plate (at (G) in FIG. 5), for downstream HTP processing. The downstream processing can include, for example, further growth of cells on selective solid media or in liquid media, separation via fluorescence-assisted cell sorting, characterization via flow cytometry, DNA extraction and NGS analysis, and/or metagenomic and phenotypic analysis of the populations.

The scale of the workflow shown in FIG. 5 is amenable to HTP. Hundreds to thousands of libraries or subsets of a single library can be processed in a single day with a sole automated platform (each well in the 6-well plate, for example, having a separate library or subset of a library), amounting to a considerable improvement over known techniques in which a human scrapes cells off a matrix using a hand-held scraper. Manual scraping comes with the additional inherent risks of sample loss, sample tracking error, and cross-contamination.

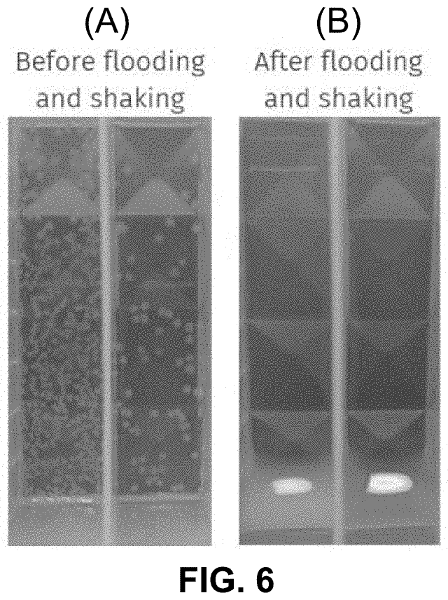

FIG. 6 shows example outcomes of a colony resuspension process, compatible with the process of FIG. 5, according to some embodiments. As can be observed in FIG. 6, colonies grown on an agar surface of a 6-well, deep-welled plate are flooded with a solution and subjected to orbital shaking (e.g., at 2,500 RPM) for a predetermined duration (e.g., between 30 seconds and 2 minutes). Upon orbital shaking, all colonies are resuspended, the cellular library is mixed together again, and transferred back to an original format (e.g., a 96-well reservoir plate), to facilitate further downstream processing and analysis (e,g., NGS analysis or phenotypic analysis).

Those skilled in the art will understand that some or all of the elements of embodiments of the disclosure, and their accompanying operations, may be implemented wholly or partially by one or more computer systems including one or more processors and one or more memory systems. Some elements and functionality may be implemented locally and others may be implemented in a distributed fashion over a network through different servers, e.g., in client-server fashion, for example. In particular, server-side operations may be made available to multiple clients in a software as a service (SaaS) fashion.

Those skilled in the art will recognize that, in some embodiments, some of the operations described herein may be performed by human implementation, or through a combination of automated and manual means. When an operation is not fully automated, appropriate components of embodiments of the disclosure may, for example, receive the results of human performance of the operations rather than generate results through its own operational capabilities.

The present description is made with reference to the accompanying drawings and Examples, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed is:

1. A method, comprising:
providing a suspension including a population of cells;
plating cells from the population of cells onto solid surfaces of each of at least one reservoir of a reservoir plate;
incubating the reservoir plate;
after cellular growth has occurred on at least one plated surface of the reservoir plate, automatically:
adding disaggregation solution to the reservoir plate,
applying a mechanical force to the reservoir plate to produce resuspended cells, and
collecting the resuspended cells.

2. The method of claim 1, wherein the population of cells includes at least one of transformed cells, transfected cells, culturable bacteria, edited microbes, or natural microbes.

3. The method of claim 2, wherein the population of cells includes the transformed cells, the method further comprising introducing DNA to cells to produce the transformed cells, prior to the plating.

4. The method of claim 1, further comprising at least one of:
adding beads to the reservoir plate prior to applying the mechanical force to the reservoir plate; or
controlling a temperature of the reservoir plate during the application of the mechanical force, such that the temperature of the reservoir plate is between 4° C. and 40° C.

5. The method of claim 1, further comprising at least one of:
automatically transferring the collected resuspended cells to a multi-well plate after the cellular growth has occurred on at least one plated surface of the reservoir plate; or
performing an automated library preparation using the collected resuspended cells.

6. The method of claim 1, wherein the disaggregation solution includes at least one of: Triton Z100, Tween-80, Urea, Pluronic™ F-68, Accumax, Accutase, Lipase, a detergent, a protease, an amilase, a cellulose, a glycerol, a microbial growth medium, an antibiotic, a viscous liquid, a positive selection agent or a negative selection agent.

7. The method of claim 6, wherein the disaggregation solution includes the microbial growth medium, the glycerol, and the antibiotic.

8. The method of claim 1, wherein the mechanical force is at least one of:
a rotational force between 200 RPM and 3,000 RPM; or
applied for a duration of between 10 seconds and 10 minutes.

9. The method of claim 1, wherein the solid surfaces of the reservoir plate comprise a liquid growth medium.

10. The method of claim 1, wherein the reservoir plate is a prepared reservoir plate, the method further comprising preparing the reservoir plate, to produce the prepared reservoir plate and prior to the plating, by applying a predetermined volume of a growth medium into each well of the reservoir plate.

11. A method, comprising:
providing a suspension including a population of cells;
plating cells from the population of cells onto solid surfaces of each of at least one reservoir of a reservoir plate;
incubating the reservoir plate;
after spores have grown on at least one plated surface of the reservoir plate, automatically:
adding disaggregation solution to the reservoir plate,
applying a mechanical force to the reservoir plate to produce a spore suspension including at least a portion of the spores, and
collecting the spore suspension.

12. The method of claim 11, wherein the solid surfaces of the at least one reservoir of the reservoir plate include an antibiotic.

13. The method of claim 11, further comprising performing an automated library preparation based on the spore suspension.

14. The method of claim 11, wherein the disaggregation solution includes at least one of: Triton Z100, Tween-80, Urea, Pluronic™ F-68, Accumax, Accutase, Lipase, a detergent, a protease, an amilase, a cellulose, or a glycerol.

15. The method of claim 11, wherein the disaggregation solution includes a viscous liquid.

16. The method of claim 11, wherein the disaggregation solution includes at least one of a positive selection agent or a negative selection agent.

17. The method of claim 11, wherein the disaggregation solution includes a microbial growth medium, glycerol, and an antibiotic.

18. The method of claim 11, further comprising adding beads to the reservoir plate prior to applying the mechanical force to the reservoir plate.

19. The method of claim 11, wherein the mechanical force is at least one of:
a rotational force between 200 RPM and 3,000 RPM; or
applied for a duration of between 10 seconds and 10 minutes.

20. The method of claim 11, further comprising controlling a temperature of the reservoir plate during the application of the mechanical force, such that the temperature of the reservoir plate is between 4° C. and 40° C.
